(19) United States
(12) Patent Application Publication
(10) Pub. No.: US 2012/0209117 A1
(43) Pub. Date: Aug. 16, 2012

(54) Surgical Measurement Apparatus and System

(75) Inventors: Alon Mozes, (US); Carlos Gil, Hallandale Beach, FL (US); Jason McIntosh, Sugar Hill, GA (US); Marc Boillot, Plantation, FL (US); Martin Roche, (US)

(73) Assignee: Orthosensor, Inc., Sunrise, FL (US)

(21) Appl. No.: 13/424,359

(22) Filed: Mar. 19, 2012

Related U.S. Application Data
(63) Continuation-in-part of application No. 11/683,410, filed on Mar. 7, 2007, now Pat. No. 8,139,029.
(60) Provisional application No. 60/779,868, filed on Mar. 8, 2006.
(60) Provisional application No. 61/597,026, filed on Feb. 9, 2012.

(51) Int. Cl. A61B 8/00; A61B 17/16; G01S 3/80

(52) U.S. Cl. 600/439; 367/127; 600/459

(57) ABSTRACT

A portable measurement system is provided including a probe, a user interface control and a receiver. The probe includes a plurality of ultrasonic transducers that emit ultrasonic waveforms for creating a three-dimensional sensing space. The user interface control captures a location and position of the probe in the three-dimensional sensing space. The receiver includes a plurality of microphones to capture the ultrasonic waveforms transmitted from the probe to produce captured ultrasonic waveforms and a digital signal processor that digitally samples the captured ultrasonic waveforms and tracks a relative location and movement of the probe with respect to the receiver in the three-dimensional ultrasonic sensing space from time of flight waveform analysis. Embodiments are demonstrated with respect to hip replacement surgery, but other embodiments are contemplated.

### CROSS-REFERENCE TO RELATED APPLICATION

This application is a continuation-in-part of U.S. patent application Ser. No. 11/683,410 filed Mar. 7, 2007 entitled “Method and Device for Three-Dimensional Sensing”, the entire contents of which are hereby incorporated by reference. This application also claims the priority benefit of U.S. Provisional Patent Application No. 61/597,026 entitled “Anatomical Pivot Point for Leg Length and Offset Calculations” filed Feb. 9, 2012, the entire contents of which are hereby incorporated by reference.

### BACKGROUND

1. Field

The present disclosure relates generally to medical interface devices, and more specifically to electronics for orthopedic instrumentation and measurement.

2. Introduction

Clinicians rely on information or media during an operative workflow. Such media may be in various visual and auditory formats. As sophisticated instruments are introduced in the clinical environment, clinicians may experience a learning curve for user interface applications. Customizing the user experience and implementing new wireless techniques into such operative workflow will advance and facilitate surgical instrument use during operative workflow.

### BRIEF DESCRIPTION OF THE DRAWINGS

FIG. 1 depicts an orthopedic tracking system in accordance with one embodiment;

FIG. 2 depicts exemplary components of the orthopedic tracking system in accordance with one embodiment;

FIG. 3A depicts a probe for presenting a media responsive to a user command during an operative workflow in accordance with one embodiment;

FIG. 3B depicts a transducer used in the probe of FIG. 3A in accordance with one embodiment;

FIG. 3C depicts a receiver that receives ultrasonic signals from the probe in accordance with one embodiment;

FIG. 3D depicts a microphone used in the receiver of FIG. 3C in accordance with one embodiment;

FIG. 4A depicts communication between exemplary components of the orthopedic tracking system in accordance with one embodiment;

FIG. 4B depicts the spatial coordinate of a probe and receiver in accordance with one embodiment;

FIG. 5A depicts a hip bone and femur and a graphical user interface of the orthopedic tracking system in accordance with one embodiment;

FIG. 5B depicts a detection of a femur head or prosthesis in the pelvis about a pivot point;

FIG. 5C depicts an offset and distance or length measured with respect to a pivot point;

FIG. 5D illustrates a process of acetabular cup placement and subsequent prosthesis placement;

FIG. 6 depicts the hip bone during a hip arthroplasty procedure and a graphical user interface in accordance with one embodiment;

FIG. 7A depicts communication between exemplary components of the orthopedic tracking system in accordance with one embodiment;

FIG. 7B illustrates signal processing of the communication in FIG. 6A in accordance with one embodiment; and

FIG. 8A-8G depicts various screen shoots of an orthopedic alignment and balance GUI in accordance with one embodiment.

### DETAILED DESCRIPTION

While the specification concludes with claims defining the features of the invention that are regarded as novel, it is believed that the invention will be better understood from a consideration of the following description in conjunction with the drawing figures, in which like reference numerals are carried forward.

In one embodiment, an apparatus can include a receiver that receives ultrasonic waveforms from a probe that emits the ultrasonic waveforms from three or more ultrasonic transducers in a three-dimensional sensing space, and a controller coupled to a memory having computer instructions which, when executed by the controller, cause the controller to digitally sample ultrasonic waveforms from the three or more microphones on the receiver to produce sampled received ultrasonic waveforms and track a relative location and movement of the probe in the three-dimensional ultrasonic sensing space from differential time of flight waveform analysis of the sampled received ultrasonic waveforms.

In a second embodiment, a probe comprises three or more ultrasonic transducers that emit ultrasonic waveforms towards a receiver that receives the ultrasonic waveforms in a three-dimensional ultrasonic sensing space where the receiver has a controller that digitally samples the ultrasonic waveforms from three or more microphones on the receiver to produce sampled received ultrasonic waveforms and tracks a relative location and movement of the probe in the three-dimensional ultrasonic sensing space from differential time of flight waveform analysis of the sampled received ultrasonic waveforms.

In a third embodiment, a portable measurement system comprises a probe having a plurality of ultrasonic transducers that emit ultrasonic waveforms for creating a three-dimensional sensing space, a user interface control that captures a location and position of the probe in the three-dimensional sensing space and a receiver. The receiver can comprise a plurality of microphones to capture the ultrasonic waveforms transmitted from the probe to produce captured ultrasonic waveforms and a digital signal processor that digitally samples the captured ultrasonic waveforms and tracks a relative location and movement of the probe with respect to the receiver in the three-dimensional ultrasonic sensing space from time of flight waveform analysis.

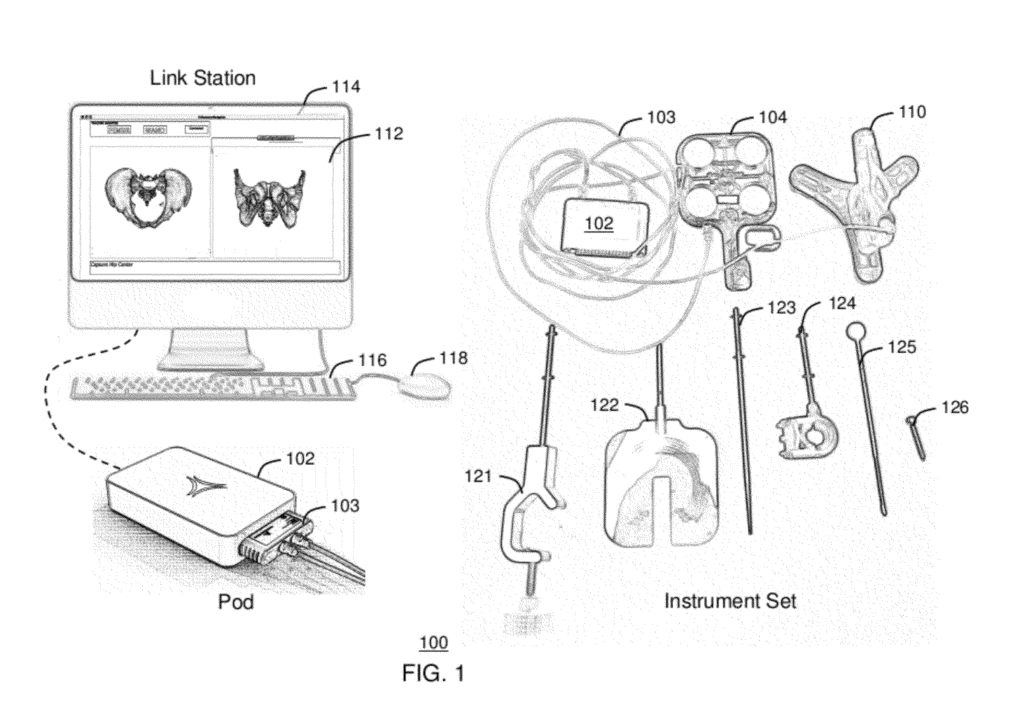

FIG. 1 depicts an orthopedic tracking system 100 in accordance with one embodiment. The tracking system 100 comprises a probe 110, a receiver 104 and a pod 102. The probe 110 emits ultrasonic waveforms for creating a three-dimensional sensing space, a probe communication link for transmitting/receiving transmission pulse data that establish a transmit time of the ultrasonic waveforms, and a user interface control 112 that captures a location and position of the probe 110 in the three-dimensional sensing space at a presentation device 114. Navigation through the user interface control 112 can be achieved using user input devices such as keyboard 116, mouse 118, and optionally the probe 110. The receiver 104 captures the ultrasonic waveforms transmitted from the probe 110 to produce captured ultrasonic waveforms. It includes a receiver communication link for relaying the captured ultrasonic waveforms to the pod 102 via a wired link 103, but optionally a wireless link can be used instead or in addition to the wired link 103.

The receiver 104 and particularly the probe 110 can be used in conjunction with a variety of instruments from an instrument set. The instrument set comprises a clamp 121 for attaching the receiver 104 or probe 110 to a surgical device, such as a remear, impactor, or other guided tool. A calibration plate 122 is used on an end of the surgical tool to calibrate the receiver 104 to the probe 110, namely mapping, a desired geometry or landmark (e.g., remear hemispherical center, rod length, rod offset, etc.) of the attached tool (e.g., remear, impactor, etc). The attachable/detachable probe tip 123 inserts within the probe 110 to mark or register (anatomical) landmarks. The probe tip position relative to the transducers on the probe 110 and corresponding geometry is predetermined and fixed. The double T end of the cam lock 125 attaches/detaches to the receiver 104 (or probe 110) when such device is to be affixed to an object (e.g., bone, operating tray, bed, stand, etc). The second end of the cam lock 124 opens/closes to clamp around the ball end of the mounting pin 125. The second end of the mounting pin 125 may be a bi-cortical screw design to insert within bone. The ball end may also be affixed to another object (e.g., operating tray, bed, stand, etc.). The cam-lock 124 and ball mount 125 permit for wide range angulation for supporting 120 degree line-of-sight between the receiver 104 and the probe 110. An example of device guidance with the probe is disclosed in U.S. patent application Ser. No. 13/277,408, filed Oct. 20, 2011, entitled “Method and System for Media Presentation During Operative Work-flow”, the entire contents which are hereby incorporated by reference.

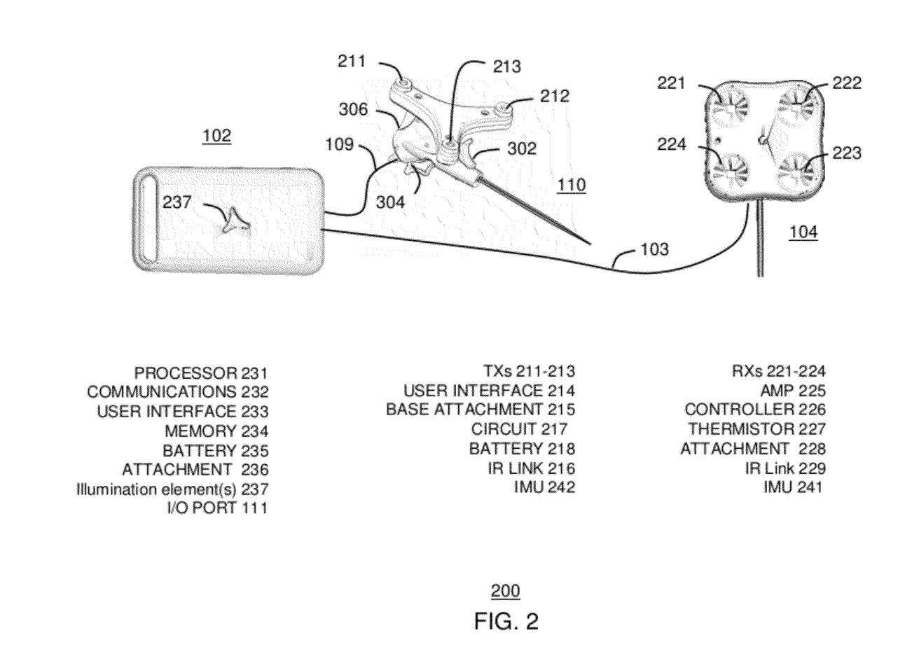

FIG. 2 depicts exemplary components of the orthopedic tracking system 200 in accordance with one embodiment. The pod 102 comprises a digital signal processor to digitally sample the captured ultrasonic waveforms and track a relative location and movement of the probe 110 with respect to the receiver 104 in the three-dimensional ultrasonic sensing space from time of flight (TOF) and differential TOF waveform analysis. The pod 102 can include a controller communicatively coupled to the probe communication link 109 and the receiver communication link 103 for synchronizing transmit and receive data functions of the digital signal processor. The pod 102 can also includes an I/O port 111 for communicating measurement data to a user interface associated with the relative location and the movement of the probe with respect to the receiver. The I/O port 111 may be a wired communication (e.g., Universal Serial Bus—USB) or wireless communication (e.g., Bluetooth or Zigbee) link. The receiver communication link 103 and the probe communication link 109 coupled to the pod 102 can be wired or wireless.

As illustrated, the system 200 comprises the pod 102, the probe 110, and the receiver 104. Not all the components shown are required; fewer components can be used depending on required functionality as explained ahead.

The pod 102 is communicatively coupled to the transmitter or probe 110 and the receiver 104 over a communication link. In the configuration shown, the pod 102 contains the primary electronics for performing the sensory processing of the communicatively coupled sensory devices. The transmitter 110 and the receiver 104 contain minimal components for operation, which permits the sensory devices to be low-cost and light weight for mounting and handling. In another configuration, the primary electronic components of the pod 102 can be miniaturized onto and/or integrated with the receiver 104 with the battery 235 and other pod components; thus removing the pod and permitting a completely wireless system.

The probe 110 can receive control information from the pod 102 over a wired connection 109 which is used for transmitting sensory signals (ultrasonic waveforms). The control information can be in the form of digital pulses or analog waveforms. Control information can be multiplexed at the pod 102 to each transmitter 110 for reducing GPIO port use. In one embodiment, the transmitter or probe 110 comprises three ultrasonic transmitters 211-213 for each transmitting signals (e.g., ultrasonic waveforms) through the air in response to the received control information. Material coverings for the transmitters 211-21 are transparent to sound (e.g., ultrasound) and light (e.g., infrared) yet impervious to biological material such as water, blood or tissue. In one arrangement, a clear plastic membrane (or mesh) is stretched taught. The transmitter or probe 110 may contain more or less than the number of components shown; certain component functionalities may be shared as integrated devices. One such example of an ultrasonic sensor is disclosed in U.S. patent application Ser. No. 11/562,410 filed Nov. 13, 2006 the entire contents of which are hereby incorporated by reference. Additional ultrasonic sensors can be included to provide an over-determined system for three-dimensional sensing. The ultrasonic sensors can be MEMS microphones, receivers, ultrasonic transmitters or combination thereof. As one example, each ultrasonic transducer can perform separate transmit and receive functions.

The probe 110 may include a user interface 214 (e.g., LED, or button) 302 and/or 304 that receives user input for requesting positional information. It can be a multi-action button that communicates directives to control or complement the user interface. With a wired connection, the probe 110 receives amplified line drive signal's from the pod 102 to drive the transducers 211-213. The line drive signals pulse or continuously drive the transducers 211-212 to emit ultrasonic waveforms. In a wireless connection, the electronic circuit (or controller) 214 generates the driver signals to the three ultrasonic transmitters 211-213 and the battery 215 of the probe 110 to provide energy for operation (e.g., amplification, illumination, timing, etc). The IR Link 216 can be an IR transmitter or photo-diode that communicates with respective elements of the corresponding IR link 229 on the receiver 104. The transmitter on either end device can send an optical synchronization pulse coinciding with an ultrasonic pulse transmission when used in wireless mode; that is, without wire line. A photo diode on the receiving end terminates the IR Link. A battery 218 can be provided for the wireless configuration if a wired line is not available to provide power of control information from the pod 102. The communications port 216 relays the user input to the pod 102, for example, when the button of the interface 214 on the probe 110 is pressed.

The probe 110 by way of control information from the pod 102 can intermittently transmit ultrasonic waves from the three (3) ultrasonic transducers. The transmission cycle can vary over a 5-10 ms interval at each of the three transmitters; each transmitter takes turns transmitting an ultrasonic waveform. The ultrasonic waveforms propagate through the air and are sensed by the microphones on the Receiver 104. The system 200 can support a system polling rate; <500 Hz. The Receiver 104 determines positional information of the probe 110 or “Wand” from range and localization of transmitted ultrasonic waveforms. The system can support short range tracking of the Receiver 104 and the probe 110 between 10 and 90 cm apart. The Receiver 104 measures the position and orientation of the probe 110 with respect to the Receiver 104 coordinate system in three-dimensions (3D) within about 120 degrees conical line of sight.

The probe 110 includes an intertial measurement unit (IMU) 242 to detect and stabilize for orientation. The inertial measurement unit 242 comprises at least one among an accelerometer for measuring gravitational vectors, a magnetometer for measuring intensity of earth's magnetic field and corresponding (north) vector, and a gyroscope for stabilizing absolute spatial orientation and position. This permits a micro-controller operation of inertial measurement unit 242 to detect abrupt physical motion of a hand-operated tool (e.g., remear, impactor, etc. attached with the probe 110) exceeding threshold limits of a pre-specified plan, and report by way of the micro-controller an indication status associated with the position and orientation that the hand-operated tool exceeds the threshold limit of the pre-specified plan.

The Receiver 104 includes a plurality of microphones 221-224, an amplifier 225 and a controller 226. The microphones capture both acoustic and ultrasonic signals transmitted by the transducers 211-213 of the transmitter 110. The frequency response characteristics of the microphone permit for low Q at a transmitter 110 resonant frequency (e.g., 40, 60, 80 KHz) and also provide uniform gain for wideband acoustic waveforms in the audio range 20 to 20 KHz. The amplifier 225 amplifies the captured acoustic signals to improve the signal to noise ratio and dynamic range. It should be noted that ultrasonic signals are also acoustic signals, yet at a higher frequency than the audio range. The controller 226 can include discrete logic and other electronic circuits for performing various operations, including, analog to digital conversion, sample and hold, and communication functions with the pod 102. The captured, amplified ultrasonic signals are conveyed over the wired connection 109 to the pod 102 for processing, filtering and analysis.

A thermistor 227 measures ambient air temperature for assessing propagation characteristics of acoustic waves when used in conjunction with a transmitter 210 configured with ultrasonic sensors. An optional IR Link 229 may be present for supporting wireless communication with the transmitter 210 as will be explained ahead. An Inertial Measurement Unit (IMU) 241 may also be present for determining relative orientation and movement. The IMU 241 includes an integrated accelerometer, a gyroscope and a compass. This device can sense motion, including rate, direction and multiple degrees of freedom, including 6 axis tilt during motion and while stationary. The IMU can be used to refine position estimates as well as detection of a pivot point from pattern recognition of circular movements approximating a hemispherical surface.

The Receiver 104 responds to ultrasonic waves transmitted by the transmitter or probe 110. If more than one probe is used, the receiver can respond in a round-robin fashion; that is, respond to multiplex transmit signals to respective transmitters 110 that emit at specific known times and within certain timing intervals. The Receiver 104 determines positional information of the transmitter 110 from range and localization of received ultrasonic waves captured at the microphones, and also from knowledge of which transmitter is pulsed. Notably, one or more transmitters 110 can be present for determining orientation among a group of transmitters 110. The pod 102 wirelessly transmits this information as positional data (i.e., translation vectors and rotational matrices) to a Display Unit. Aspects of ultrasonic sensing are disclosed in U.S. patent application Ser. No. 11/839,323 filed Aug. 15, 2007, the entire contents of which are incorporated by reference herein. An IMU 241 with operation similar to the IMU 242 on the probe 110 can be present on the receiver 104. The Pod 102 can comprise a processor 231, a communications unit 232, a user interface 233, a memory 234 and a battery 235. The processor 231 controls overall operation and communication between the transmitter 110 and the receiver 104, including digital signal processing of signals, communication control, synchronization, user interface functionality, temperature sensing, optical communication, power management, optimization algorithms, and other processor functions. The processor 231 supports transmitting of timing information including line drive signals to the transmitter 110, receiving of captured ultrasonic signals from the receiver 104, and signal processing for determination of positional information related to the orientation of the transmitter 110 to the receiver 104 for assessing and reporting cut angle information.

The processor 231 can utilize computing technologies such as a microprocessor (uP) and/or digital signal processor (DSP) with associated storage memory such a Flash, ROM, RAM, SRAM, DRAM or other like technologies for controlling operations of the aforementioned components of the terminal device. The instructions may also reside, completely or at least partially, within other memory, and/or a processor during execution thereof by another processor or computer system.

The electronic circuitry of the processor 231 (or controller) can comprise one or more Application Specific Integrated Circuit (ASIC) chips or Field Programmable Gate Arrays (FPGAs), for example, specific to a core signal processing algorithm or control logic. The processor can be an embedded platform running one or more modules of an operating system (OS). In one arrangement, the storage memory 234 may store one or more sets of instructions (e.g., software) embodying any one or more of the methodologies or functions described herein.

The communications unit 232 can further include a transceiver that can support singly or in combination any number of wireless access technologies including without limitation Bluetooth, Wireless Fidelity (WiFi), ZigBee and/or other short or long range radio frequency communication protocols. This provides for wireless communication to a remote device 104 (see FIG. 1). An Input/Output port within the communications unit 232 permits portable exchange of information or data, for example, by way of Universal Serial Bus (USB).

The memory 234 stores received ultrasonic waveforms and processing output related to tracking of received ultrasonic waveforms and other timing information, state logic, power management operation and scheduling. The battery 235 powers the processor 231 and associated electronics thereon and also the transmitter 110 and the receiver 104 in the wired configuration.

The user interface 233 can include one or more buttons to permit handheld operation and use (e.g., on/off/reset button) and illumination elements 237 to provide visual feedback.

In a first arrangement, the receiver 104 is wired via a tethered electrical connection (109 and 103) to the transmitter 110. Timing information from the pod 102 tells the transmitter 210 when to transmit, and includes optional parameters that can be applied for pulse shaping and noise suppression. The processor 231 on the pod establishes Time of Flight measurements according to the timing with respect to a reference time base in the case of ultrasonic signaling. One example of pulse shaping is taught in U.S. Pat. No. 7,414,705 the entire contents of which are hereby incorporated by reference. In a second arrangement, the receiver 104 is wirelessly coupled to the transmitter 110 via an optical signaling connection. The infrared transmitter 216 on the transmitter 110 transmits an infrared timing signal with each transmitted pulse shaped signal. The infrared timing signal is synchronized with the transmitting of the ultrasonic signals to the receiver 104. The receiver 104 can include the IR Link 229 (e.g., IR emitter or photo diode) which the pod 102 monitors to determine when the infrared timing signal is received. The pod 102 can synchronize infrared timing information to establish Time of Flight measurements with respect to a reference transmit time. The infrared transmitter and photo diode establish transmit-receive timing information to within microsecond accuracy.

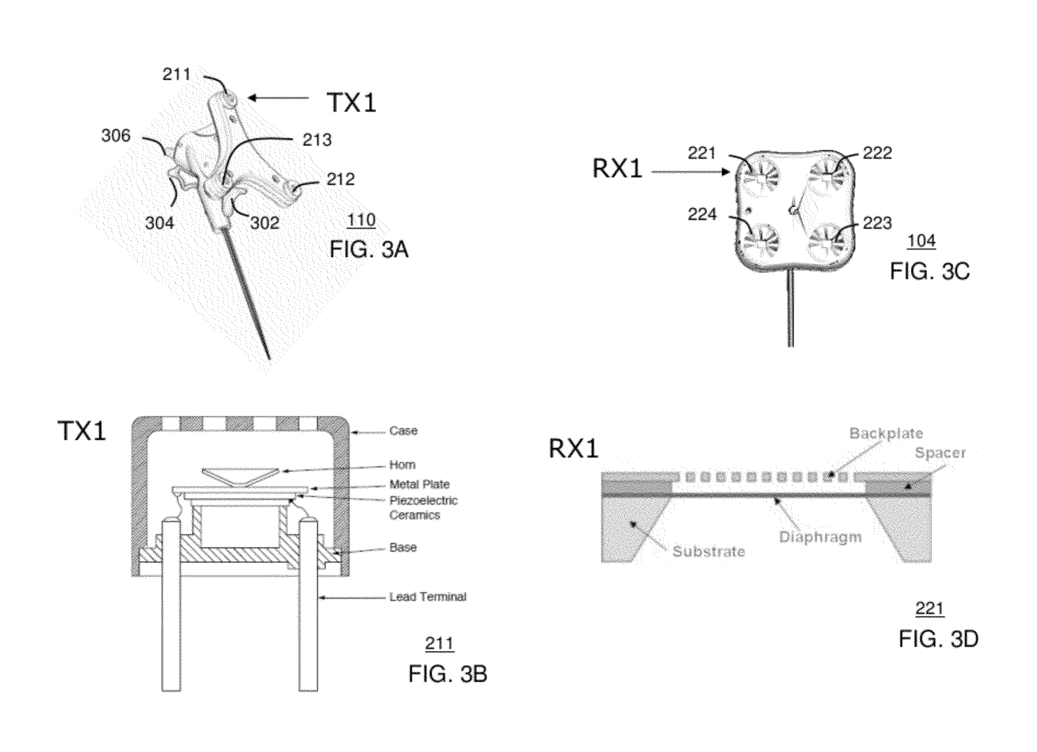

FIG. 3A depicts the probe 110 for presenting a media responsive to a user command during an operative workflow, which may include navigating a GUI of the link station or registering anatomical points for creating a reference coordinate system. The probe 110 is one embodiment of the transmitter 110 shown in FIG. 2. It is configured for user control by way of a three-way switch 302. The three-way switch provides for leftward indexing, center button press, and rightward indexing. The probe also includes a release lever 304 for coupling to a probe pointer or probe plate as described above; that is, for capturing anatomical information and/or reporting geometric information. The connector 306 comprises a part of the probe communication link 109 for wired configuration. The probe also includes an illumination element (e.g., LED) to convey status. As an example, the led intermittently flashes green to indicate working status, turns red in certain communication conditions (e.g., out of line of sight, communication protocol errors, etc.), and stays green when the switch 302 is activated, for example, upon capturing a landmark. The probe 110 provides user control to capture points or planes with respect to the location of the receiver 104 and optionally with respect to first tracker/transmitter and the second tracker/transmitter (not shown). Aspects of GUI navigation by way of the probe 110 are disclosed in U.S. patent application Ser. No. 12/900,662 filed Oct. 8, 2010, the entire contents of which are incorporated by reference herein.

With respect to FIG. 3B, the ultrasonic transmitters 211, 212, and 213 can be constructed using an open structure type ultrasonic sensor. A multiple vibrator can be fixed elastically to a base. This multiple vibrator is a combination of a resonator or horn and a vibrator, which is composed of a metal sheet and a piezoelectric ceramics sheet. The resonator is conical in order to efficiently radiate the ultrasonic waves generated by the vibration and also in order to effectively concentrate the ultrasonic waves at the central part of the vibrator.

Referring to FIG. 3C, the receiver can be constructed from a plurality of receivers or microphones 221, 222, 223, and 224. Each of the microphones can be constructed as illustrated in FIG. 3D where microphone 221 is shown in greater detail. For example, the Knowles SPM0404UD5 “Si-sonic” ultrasonic sensor can be used as the “receiver” in the hip Alignment System described herein. It is a surface mount wide-band ultrasonic sensor for applications in hand held communication devices and positioning sensing. The SiSonic is a silicon-based MEMS microphone with robustness against shock and low sensitivity to temperatures. It belongs to the group of sensors known as condenser microphones. As shown in FIG. 3D, there are two electrodes involved, which form a capacitor. The back plate, which is the static electrode, is highly perforated and hence “acoustically transparent”. The flexible electrode—the diaphragm—is suspended and susceptible to pressure changes in the air and can vibrate when exposed to sound. The vibrations of the diaphragm actually modulate the value of the capacitor itself. These modulations are electrically amplified and used for the measurement of ultrasonic waves.

The entire SiSonic SPM0404UD5 condenser microphone is a square die manufactured entirely from silicon. The operational temperature range is −40° C. to +105° C. and therefore far wider than standard Electret Condenser Microphone (ECM) ratings. Under specified environmental conditions the standard deviation of the sensitivity is maximum 1 dB. After test conditions are performed, the sensitivity of the microphone does not deviate more than 3 dB from its initial value.

The ultrasound based tracking technology determines spatial position through measurement of ultrasonic waves. As shown in FIG. 1, for a single distance measurement, the transmitter transmits an ultrasonic pulse at a known time, t0, which is later received at the receiver at a known time, t1. The Time of Flight measurement (TOF=t1−t0) is calculated between the transmitter and the receiver sensor. The distance is calculated from the equations below, given the TOF, speed of sound in air and the temperature. Each paired transmitter and receiver provides a 1-dimensional spatial measurement; namely, distance.

D=dTOF*c, where

c=331.5+0.607t(m/s) where t=temperature(° C.) EQ 1

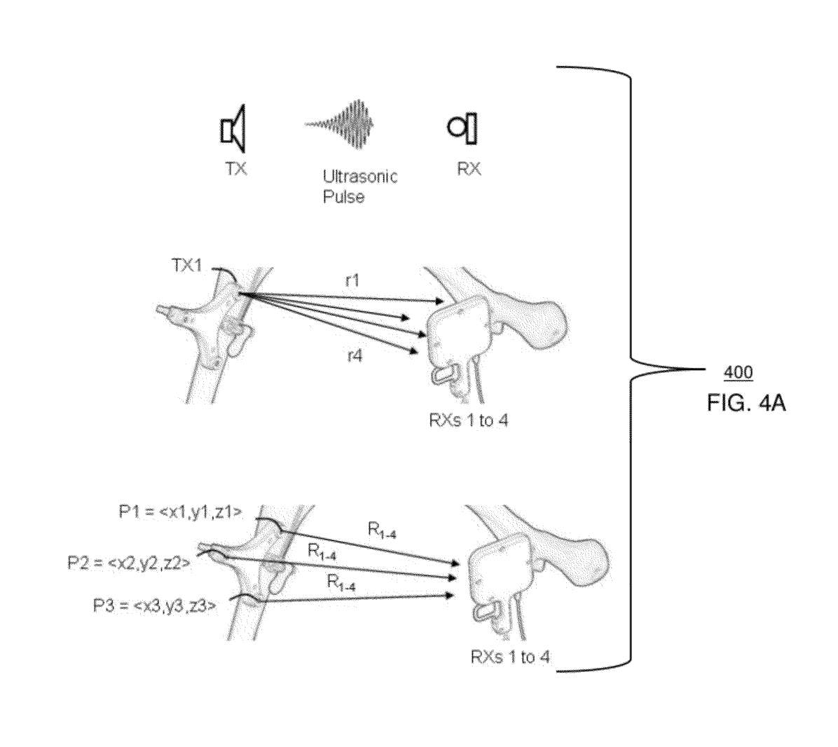

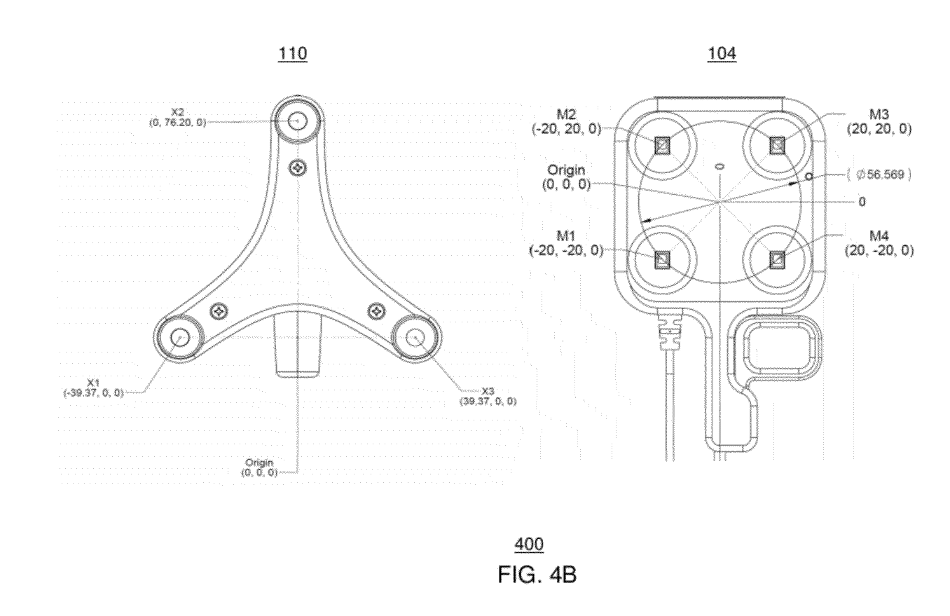

Referring to FIG. 4A, a system 400 illustrates a paired transmitter and receiver and particularly in the context of bone elements as might be used in surgery or an operative work flow. The ultrasound based tracking technology determines angular orientation through the trilateration of multiple distance measurements between multiply paired transmitters and receivers. Providing three transmitter-receiver pairs provides three-dimensional spatial measurements (3 degrees of freedom tracking) as distances can be calculated with EQ1 between each pair. The multiple distance measurements permit the determination of orientation and angular relationships between devices. A fourth receiver provides for an over-determined system and provides for numerical boundary checks.

Referring FIG. 4B, a probe on the left can have 3 transmitters and a receiver on the right can have 4 microphones. The physical layout geometry of the sensor configurations on each respective device (probe, receiver, etc.) establishes known geometries for determining angular relationships. The geometry establishes fixed distances between each sensor relative to its neighbors on the same device; that is, the sensors are places at precise known locations on the device and physically remain there. The distances are expressed as point locations in a local device reference coordinate system, as shown in FIG. 4B. Accordingly, the arrangement shown in FIG. 4B provides 6 degrees of freedom tracking between two devices in three-dimensional space; that is, given the known geometry of the 3 transmitters on the Probe and the known geometry of the 3 or more receivers (hereinafter microphones) on the Receiver.

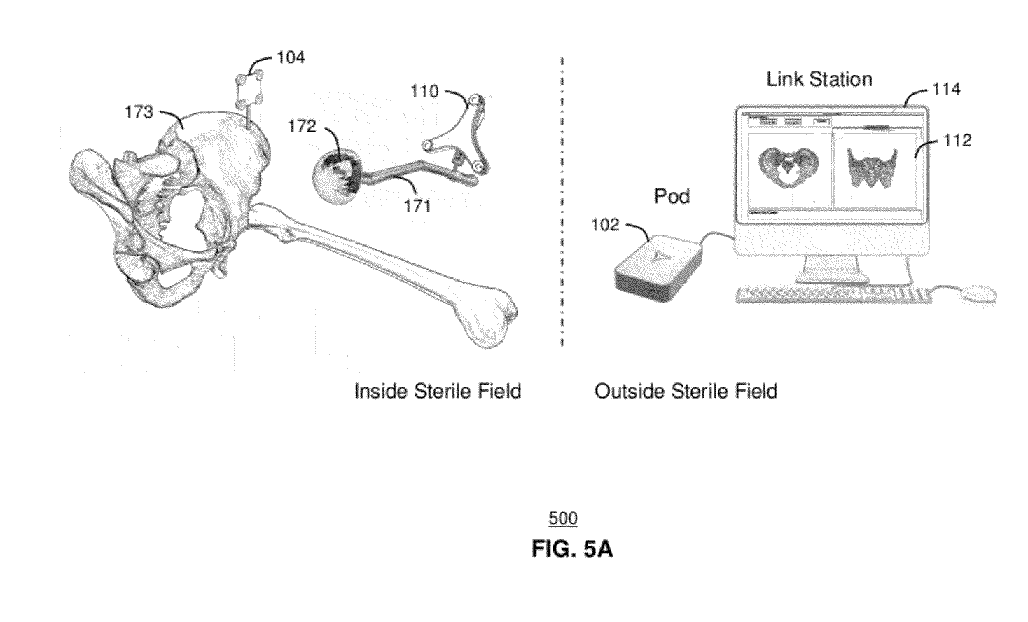

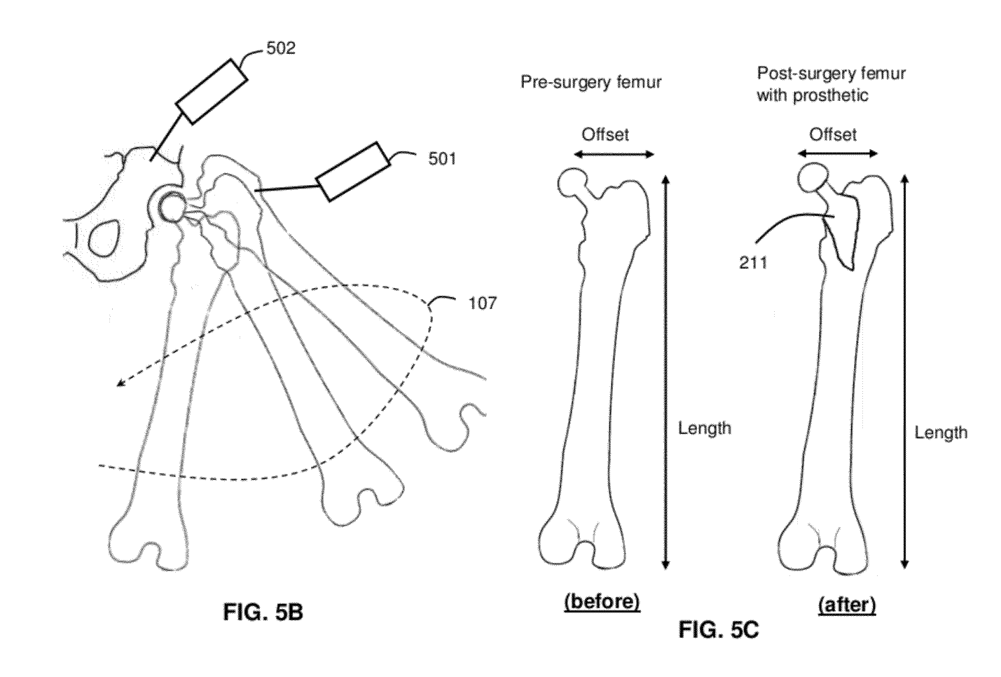

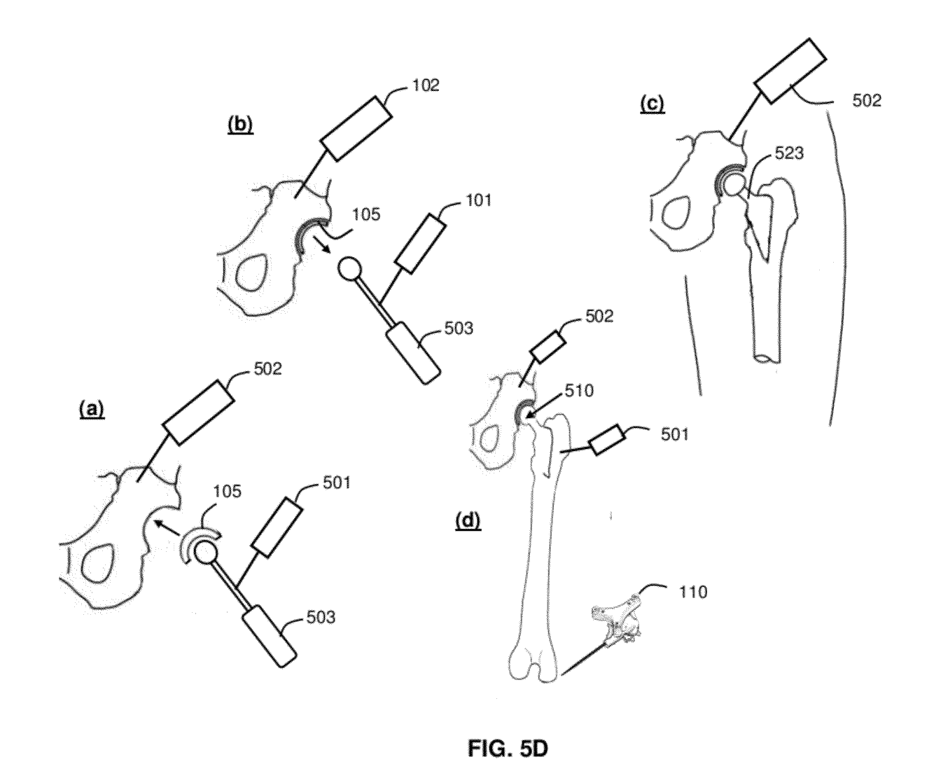

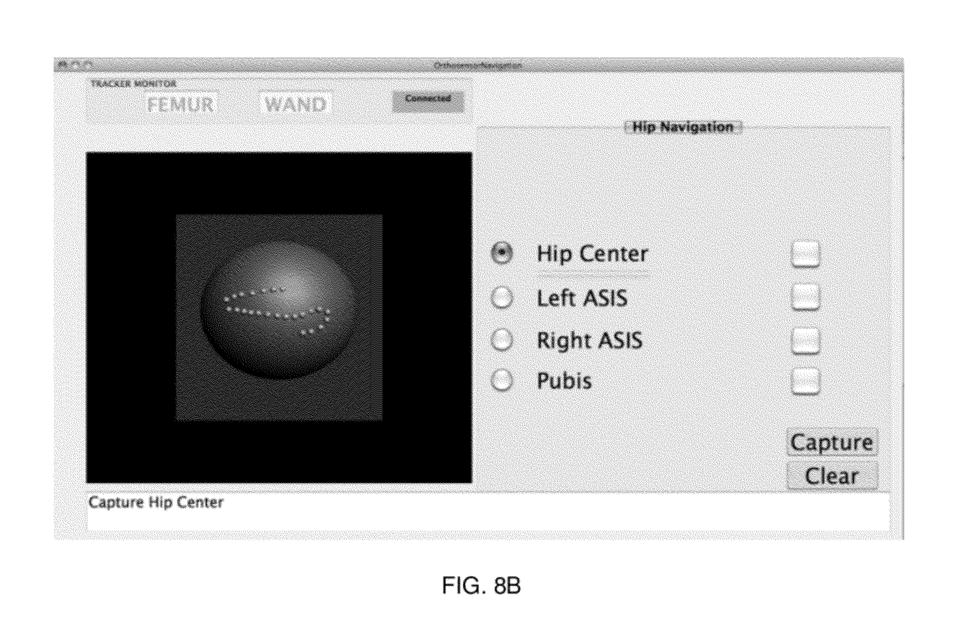

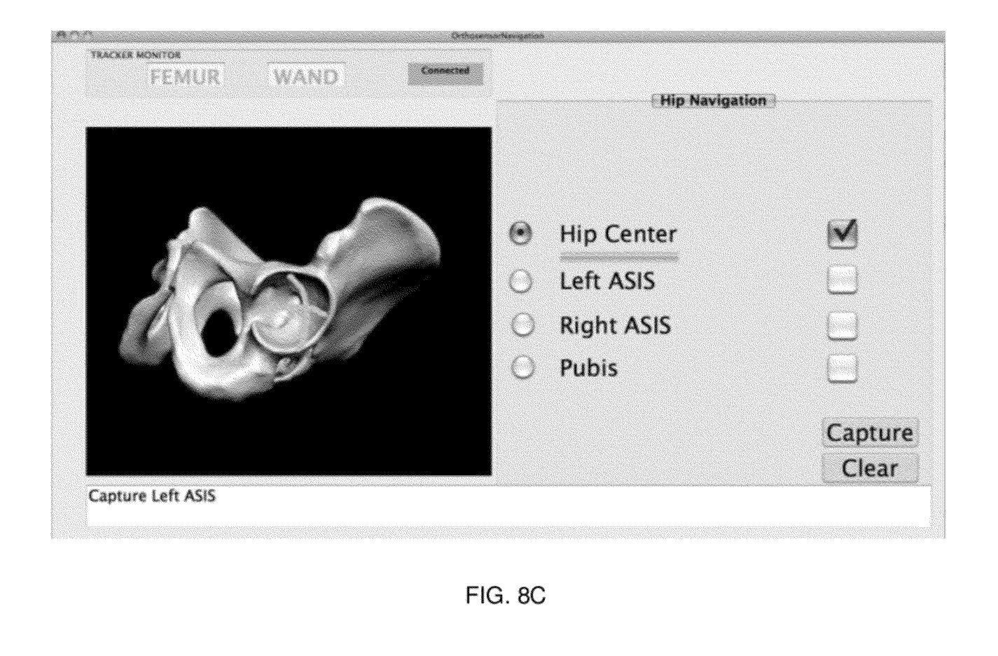

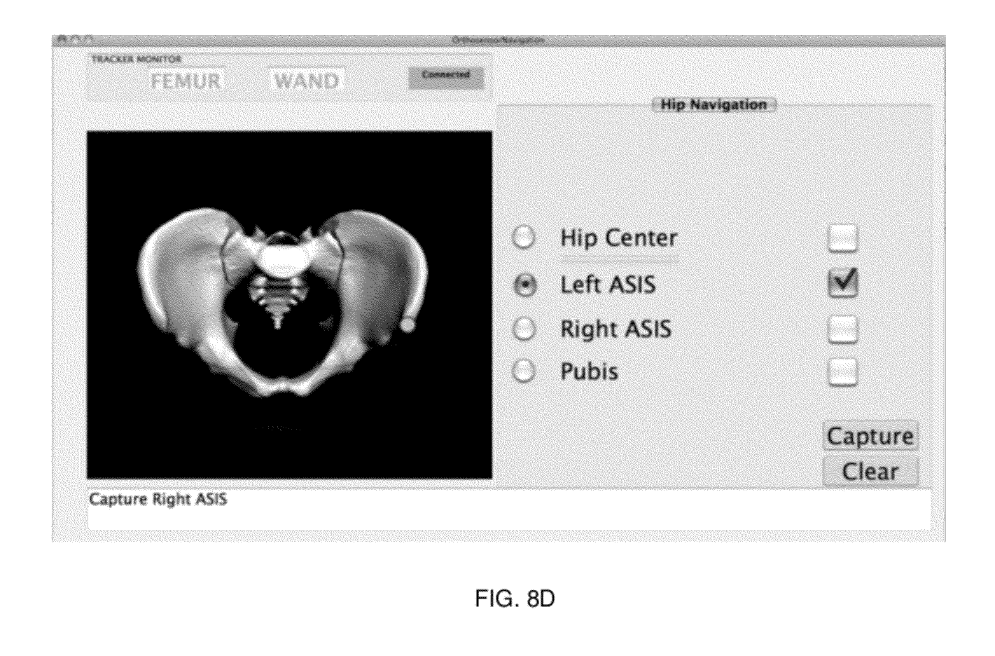

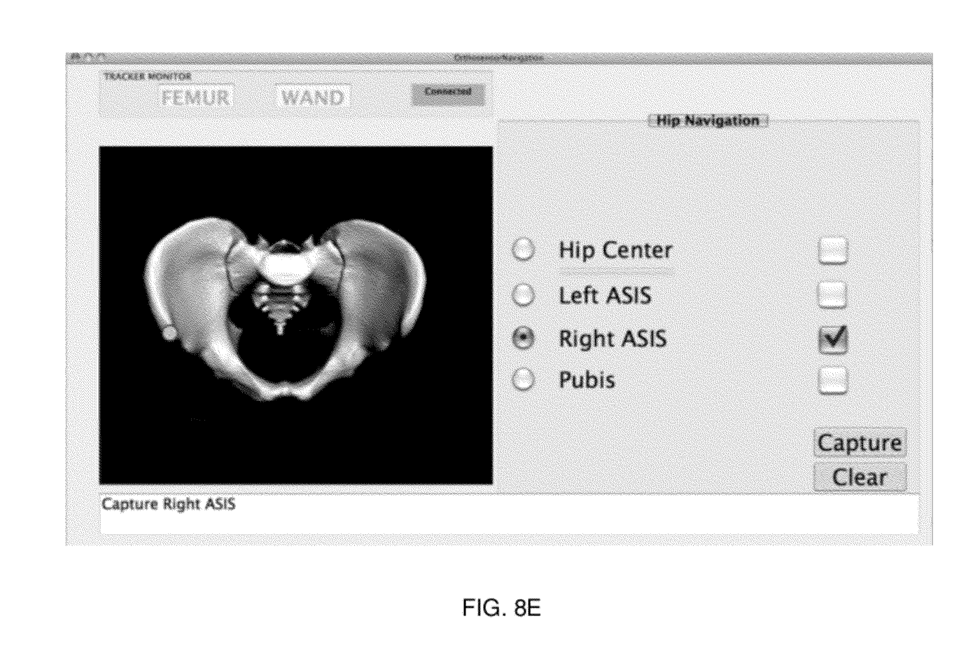

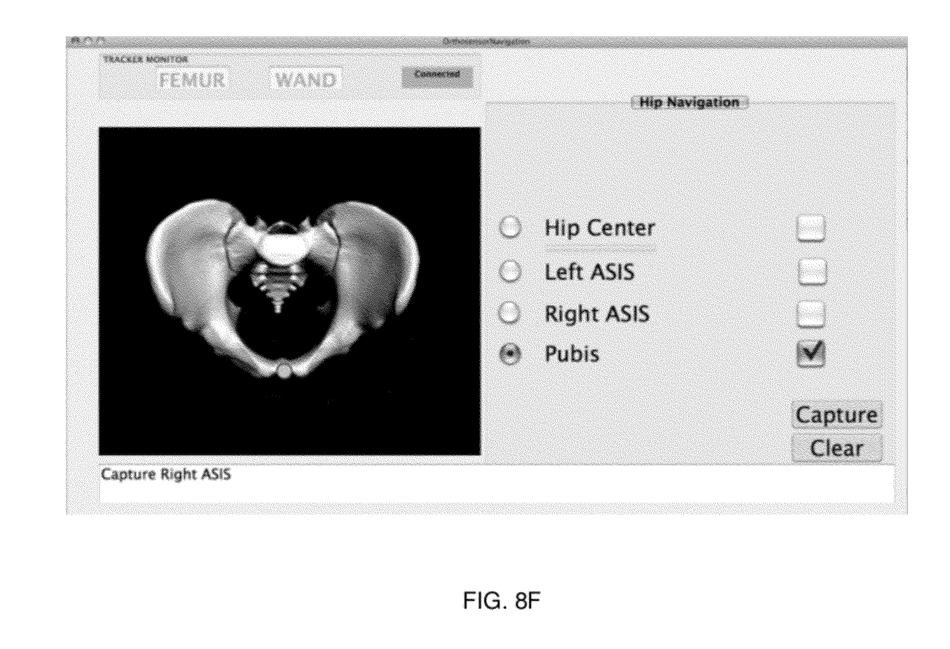

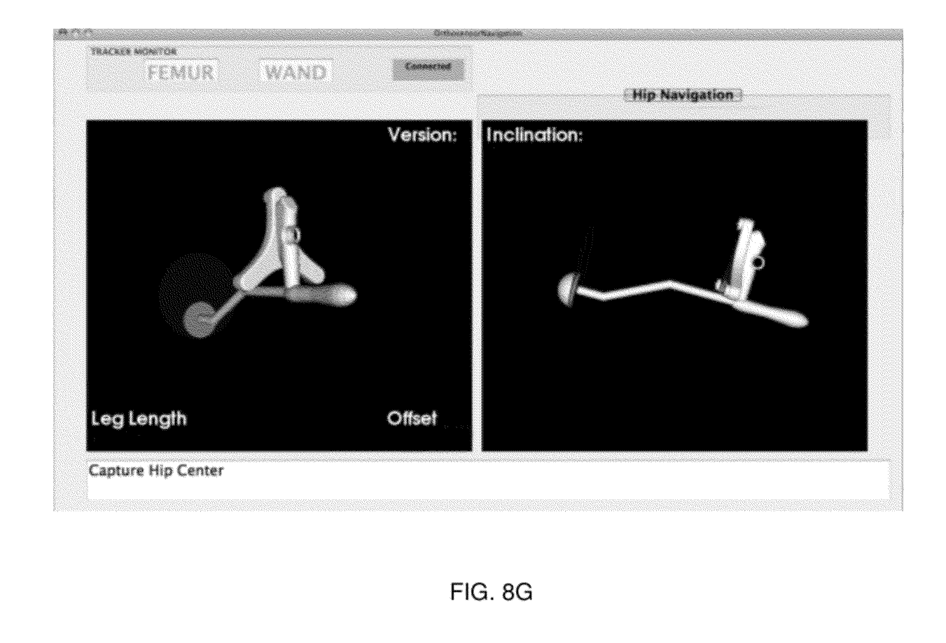

Referring to FIG. 5, the graphical user interface (GUI) 112 of an orthopedic tracking system 500 is shown on the presentation device 114 in the context of a hip replacement operative workflow. The GUI 112 receives by way of the pod 102 a command from the probe 110 during high-resolution position tracking of the probe 110, and presents a media that corresponds to the user interface command. The GUI 112 exposes, or adjusts, a state of the media (e.g., GUI check box, text box, radio button, text instruction, etc.) responsive to a pressing of the probe switch 302 (See FIG. 3A). The media can be at least one among audio, image, video, and text. For instance, upon the user pressing the switch 302 on the probe, the pod 102 directs a user command to the GUI 112 to alter a state of a user interface component as illustrated in FIG. 8. As an example, the GUI may illuminate an element of a hip such as a hip center, left ASIS, right ASIS, or Pubis as in FIG. 8(b) to indicate a next operation workflow step. The user, upon placement of a probe plate onto a desired measurement area, presses the center button of the switch 302 to capture the landmark plane, and the GUI marks component with a checkmark as in FIG. 8(c)to indicate successful capture. The GUI 112 can automatically scroll to the next GUI element such as the Left ASIS as in 8(d, e, and f) and indicate a successful capture. Aspects of GUI navigation by way of the probe 110 are disclosed in U.S. patent application Ser. No. 13/164,396 filed Jun. 20, 2011, the entire contents of which are incorporated by reference herein.

During operative workflow, the user can index the three-way switch 302 left or right to navigate forward or backward over GUI components as well as pages of a tab menu of the GUI. As illustrated, a hip nav page is displayed in the tab menu. Each page of the tab menu is associated with an operative workflow, for example, as shown for hip replacement surgery. In the exemplary illustration, the tab menu can present various pages (Patient Info, HIP Nav, Tool Nav, HIP-Leg Alignment) corresponding to an operative workflow of a Hip replacement. The operative workflow and accordingly the GUI 112 can be designed specific to an orthopedic procedure (e.g., knee, hip and spine) with pages of the tab menu similarly designed. The pod 102 thus presents the media according to a customized use of the probe during an operation workflow. It permits navigating a menu system of a Graphical User Interface via the tracking of the probe relative to the receiver. Furthermore, the pod 102 can recognize an operation workflow and report measurement data from the probe associated with the operation workflow. As one example, upon moving the probe in a circular pattern the device can automatically detect femur head identification and proceed to the corresponding user component and page of the tab menu. Aspects of detecting a femur head are disclosed in U.S. patent application Ser. No. 12/853,987 filed Aug. 10, 2011, the entire contents of which are incorporated by reference herein. Aspects of pattern recognition using neural networks and hidden Markov models in ultrasonic sensing applications for recognizing user interface gestures are also disclosed in U.S. patent application Ser. No. 11/936,777 filed Nov. 7, 2007, the entire contents of which are incorporated by reference herein.

Referring again to FIG. 4A, position determination by way of the ultrasonic tracking system is performed using the trilateration methodology (spherical positioning) where the absolute Time-of-Flights (TOF) between each of the transmitter/receiver pairs on each device is measured. The trilateration principle can be explained as follows. Given that the transmitters and receivers are synchronized, i.e. their clocks have precise time, the TOF is measured at the transmitter TX1 of unknown position (x, y, z), tk, to each of the four microphones k on the Receiver. The TOFs are then multiplied by the speed of the propagating wave, c, to obtain a set of ranges, rk, from the transmitter to each individual microphone, mk. Each range, rk, is captured in the equation of a sphere of radius rk centered at each microphone at position (xk, yk, zk) (EQ. 2). A system of N equations (N 3) is derived whose solution provides the transmitter position (x, y, z).

\sqrt{(xₖ−x)²+(yₖ−y)²+(zₖ−z)²}=rₖ EQ 2

The trilateration method of multiple TOFs generates a unique position <x,y,z> of a single transmitter, as shown by the middle figure of FIG. 4A. The trilateration method is thereafter applied to each transmitter TX1, TX2, and TX3 of the (Probe) device for calculating the spatial locations P1, P2 and P3 for the respective transmitters as shown in the bottom figure of FIG. 4A. This process can be repeated for each tracking device, where each set of points are spatial coordinates with respect to a reference coordinate system of the Receiver.

With bone surgeries involving hips, knees or elbows, determination of rotation and translation will be used. The spatial coordinates for each transmitter of the device are used to create a transformation matrix that represents the rotation and translation (position) of the device. This transformation matrix establishes the orientation (angle) and position (location) of the device relative to the Receiver.

### Spatial Data:

Three-Dimensional Points (<x,y,z> Cartesian points)
Matrix Rotations (3×3 matrix of <x,y,z> points)
Translation Vectors (3×1 vector of <x,y,z> points)
   The 4×4 transformation matrix includes an interior 3×3 rotation matrix R and a 1×3 translation vector P.

$$[ {[ \begin{matrix}Px \\ Py \\ Pz\end{matrix} ]}^{1} {[ \begin{matrix}\begin{matrix}Rxx \\ Ryx\end{matrix} \\ Rzx\end{matrix}}^{0} {\begin{matrix}\begin{matrix}Rxy \\ Ryy\end{matrix} \\ Rzy\end{matrix}}^{0} {\begin{matrix}\begin{matrix}Rxz \\ Ryz\end{matrix} \\ Rzz\end{matrix} ]}^{0} ]$$

The 3×3 rotation matrix identifies the rotation about the devices local X, Y and Z coordinate axes, as noted below.

$${H}_{R} = [ \begin{matrix}M & 0 \\ 0 & 1\end{matrix} ] \qquad Where \text{:} M is the rotation matrix$$

$$M x ( θ ) = [ \begin{matrix}1 & 0 & 0 \\ 0 & cos ( θ ) & - sin ( θ ) \\ 0 & sin ( θ ) & cos ( θ )\end{matrix} ] \qquad Where \text{:} Theta is the rotation around the X \text{-} Axis$$

$$My ( θ ) = [ \begin{matrix}cos ( θ ) & 0 & sin ( θ ) \\ 0 & 1 & 0 \\ - sin ( θ ) & 0 & cos ( θ )\end{matrix} ] \qquad Where \text{:} Theta is the rotation around the Y \text{-} Axis$$

$$Mz ( θ ) = [ \begin{matrix}cos ( θ ) & - sin ( θ ) & 0 \\ sin ( θ ) & cos ( θ ) & 0 \\ 0 & 0 & 1\end{matrix} ] \qquad Where \text{:} Theta is the rotation around the Y \text{-} Axis$$

The 1×3 translation matrix P identifies the (x,y,z) location of the device, as defined by its local coordinate system origin, relative to the origin of the Receiver coordinate system.

The Pod (102) can generate the Spatial Data in accordance with the trilateration TOF analysis of the received ultrasonic signals. The calculated spatial data from the Pod is then communicated over a USB connection, for example, to a computer and presentation device 114 (see Link Station of FIG. 1). The data processing is a one-way communication from the Pod to the Computer. The computer does not control the Sensor Set devices or the Pod.

The Pod also provides error codes with the spatial data that report the data integrity and device status indications. It does this by way of a device driver (dynamic library) on the computer that received the data communication. The computer provides error control and renders the data to the display through the GUI.

The instrument set (of FIG. 1) can include a number of probe pointers and plates. The Pointer and Plate attachments to the Probe 110 are used for capturing and reporting anatomical landmarks and for reporting bone cut angles and distances. Both the Pointer and Plate have inherent geometries that specify where the Plate surface is, or where the Pointer tip is with respect to the Probe. The Pointer is specified by four spatial locations (each a specified coordinate as illustrated in the FIG. 4B:
P1—Pointer Tip <x,y,z>

The Plate is specified by four spatial locations (each a specified coordinate as listed below):
C1—Center <x,y,z>
C2—Medial <x,y,z>
C4—Lateral <x,y,z>
C3—Front <x,y,z>

The spatial coordinates of the Plate and/or Pointer are measured during manufacture and saved to a memory in the instrument device cable. The Pod retrieves these coordinates as well as calibration data from the memory of the device cable once connected. The Computer then retrieves the Plate and/or Pointer coordinates from the Pod once connected. The Computer then makes use of the Plate and/or Pointer coordinates within the GUI as anatomical landmarks are captured for creating the reference bone coordinate systems and for reporting bone cut angles and resection depth measurements or for placement of cups in a hip center for use in adjusting.

Broadly stated, a system and method is provided for touchlessly resolving a pivot point and other measurements in a 3 dimensional space. This can be particularly suited for situations where one end of a rigid object is inaccessible, but remains stationary at a pivot point, while the other end is free to move, and is accessible to an input pointing device. The system as noted above comprises a probe or wand and a receiver that are spatially configurable to touchlessly locate the pivot point without direct contact. The wand and receiver uniquely track each other's relative displacement to geometrically resolve the location of the pivot point. The pivot point can be a hip joint and a rigid object can be a femur bone with one end at the hip joint and the other end free to move.

The embodiments will be further described below in the context of a Hip replacement. Hip replacement surgery is a very successful procedure to alleviate pain from an arthritic hip joint. A successful outcome requires the resurfacing of the pelvic acetabular cup and the diseased femoral head. The Hip joint articulates in a ball and socket configuration, and the hip joint replacement incorporates an artificial acetabular cup, femoral stem, various modular neck and heads. The surgeon removes the diseased portions of the joint, and attempts to create a stable joint by appropriate positioning of the acetabular cup and femoral components. He mainly does this through visual landmarks and surgical experience. Common errors include a poorly positioned acetabular cup as it relates to version, inclination, depth, and position. This can lead to dislocations, subluxations, component edge loading, early poly wear, cup loosening and migration. The femur can be malpositioned as well as it relates to version, position, varus-valgus orientation. This can lead to hip instability—dislocation, or a tight hip leading to pain, limp, and limited motion. The use of modular heads and necks are in response to improving hip stability. A leg can be inappropriately lengthened or the hips offset may be over- or under tensioned leading to a failure of the surgery.

Described herein is a surgical approach integrating ultrasonic (US) technology positioning as it relates to implant preparation and implantation of the prosthesis. The system can be integrated with image or imageless systems. The US sensors can be attached to bony landmarks for tracking and attached to standard instruments to allow appropriate bony preparation. The surgeon pre-operatively images the hip and determines the implant sizing and positioning. The key element is defining the center of hip rotation that is present and what hip center he wants to achieve intra-operatively. The surgeon positions the patient as it relates to his approach. A pin can be mounted in the ipsilateral ASIS. The pin is stabilized to the pelvis. Knowing the positioning of the pelvis, a sensor is mounted to the pin. The pelvis and acetabulum can be registered to a pre-op image (e.g., digital X-ray, Computer Assisted Tomography (CAT), Magnetic Resonance Imaging (MRI), etc.) or an imageless system.

Referring now to FIG. 5A, a graphical user interface 112 presenting measurement media of the orthopedic tracking system is depicted in accordance with one embodiment. As previously indicated, the pod 102 can include a display unit 114 to render 2D/3D visual information corresponding to the orientation and position of transmitters or probes or trackers 110 with respect to the receiver 104 coordinate system, and furthermore, any devices thereto mounted. For example, as described in U.S. Provisional Patent Application 61/498,647, the contents of which are hereby incorporated by reference in entirety, and which priority reference is hereto claimed, a device attachment can be mounted to the probe 110 to provide bone cut angle information, or a probe attachment can be thereto mounted to provide spatial position information. In the illustration shown the probe 110 is attached to a surgical tool 171, which for example, may be a remear with a hemispherical end 172. The tool 171 is placed within the acetabulum of the pelvis 173 (which holds the femur head and permits leg rotation and movement) wherein the hemispherical end reams out cartilage for placing a prosthetic cup. The hemispherical end 172 data (center sphere coordinates and inclination/inversion with respect to the tool 171 are predetermined either through known device geometries or through the mapping as explained above in FIG. 1 using the calibration plate 122. The link station with attached pod 102 and receiver 104 track the location of the probe 110, and accordingly, the surgical tool 171 and its physical geometries and features (i.e., where the hemispherical end 172 is with respect to the acetabular cup; information which may be stored in a memory either on the pod 102 or a communicatively coupled link station that specifically relates the tool 171 orientation to the probe 102 orientation. Thus the orthopedic tracking system 100 serves as a tracking and measurement device to assess anatomical features and spatial distances between anatomical points for guiding surgical tools with specific features

In another arrangement, as part of the surgical work flow, the probe 110 may be thereafter attached to an impactor (e.g. 171) with a temporarily mounted prosthetic cup (e.g., 172) that is guided into position in the acetabular cup of the pelvis 173. In such arrangement, the link station 114 by way of the GUI 112 permits the user to guide the cup 172 to a center of the acetabular cup (hip joint) in the pelvis 173 with a known inversion angle and inclination angle with respect to the reference coordinate system of the pelvis. Briefly, the reference coordinate system is created from the previous registration of pelvic anatomical landmarks (i.e., left ASIS, right ASIS, pubis, etc) by way of the probe 110 pointer tip marking these anatomical landmarks. This coordinate system also serves as the reference for the acetabular cup center which is referenced for determination of leg offset and leg length.

As one example of capturing the acetabular cup center, referring to FIGS. 5B and 5C a first sensor 502 (e.g., receiver or probe) attached to the pelvis is provided for tracking pelvic movement. A second sensor 501 (receiver or probe) is attached to a prosthetic component (femur head/neck) to track femur bone movement. This utilizes external sensor mount position (501 & 502) in conjunction with method to determine pivot point for contralateral leg (the leg opposite to that which will be operated on, or the “operative leg”) for comparison and to assist in establishing or re-establishing normal biomechanics, and to store as a reference point for any future surgery including primary surgery on the contralateral hip or knee joint; primary surgery on the knee joint of the operative leg; or revision surgery on the hip joint of the operative leg. An example of capturing and localizing the femur head center of the acetabular cup is disclosed in U.S. patent application Ser. No. 12/900,955, filed Oct. 8, 2010, entitled “Orthopedic Method and System for Mapping an Anatomical Pivot Point”, the entire contents of which are incorporated by reference in entirety.

In an anterior approach, the contra-lateral ASIS (anterior superior iliac spine) can be mounted, and the hip joint defined by the rotation method described here. By knowing what the non-diseased hip joint center and leg length are, this information can be utilized to compare the operated hip outcome intra-operatively, and adjustments made as necessary to achieve balanced hips and leg lengths/offset.

The pelvis is registered by collecting a set of points on the pelvis, and in the acetabular cup, for example, as described using probe pointer registration The probe 110 tracker wirelessly sends this information to the receiver 104. A particular way to register the cup is to remove the femoral head from the joint, and utilize a sensorized initial cup trial (105 as illustrated in FIG. 5D) that houses multiple pins that are able to be deployed in a unit collecting various points on the cup and registering it in one step. The sensors in the cup may be isolated IMU 242 components or sub-components thereof (e.g., accelerometer, magnetometer) for localizing directional vectors of the acceleration or magnetic poles.

A strap (not shown) can be securely attached to the distal thigh and if the hip joint is mobile, the center of the hip is registered with circumductive movements. (See FIG. 5B). The surgeon can use this center or having predicted where he wants the new hip center to be, the surgeon can utilize the software to define the new position. A pin can be mounted in the greater trochanteric region of the femur, or attached to the initial femoral trial stems and thus avoid pins in the femur. Points can be registered on the femur and incorporated into the pelvic receiver to utilize inter-incisional navigation and avoid line of site issues. By knowing the depth, leg offset and leg length, the appropriate head and neck length and sizes can be trialed to define the best fit. (See FIG. 5C) In FIG. 5C on the right, a pin is shown extending from the trial and a US sensor attached to it. The preparation of the acetabular cup requires reaming the cup to the correct depth, inclination and version as well sizing the most stable cup size and liner. The US sensor can be attached to any handle that handles a powered reamer that is held by the surgeon's hand or robotically guided to allow navigation of the reaming process to ensure appropriate depth, version and inclination. By interfacing with the GUI, the surgeon can adjust the angle and force used based on preset parameters of what is considered the optimal angles and depth. The software will help guide the procedure in a dynamic real time way with immediate feedback to the surgeon.

Once the cup is reamed under guided navigation, the surgeon may now attach the final acetabular cup that will be the final implant. The US sensor as it is attached to the insertion handle, now guides the insertion of the cup, and as the surgeon impacts the implant in, with known reported inversion, inclination and depth through the software on the GUI that depicts when he has achieved optimal implantation. (See FIG. 5D (a-d). The surgeon can now remove the insertion handle from the acetabular component and insert the trial poly insert as shown in FIG. 5D (b).

The femoral stem preparation now begins. The femoral canal is broached and trial stems are inserted to achieve appropriate canal fill and stability while appropriate version and depth are defined. Modular head and neck are attached to the stem and the hip joint is reduced. By having an ultrasonic (US) tracker on the greater trochanter or attached to the lateral aspect of the trial itself, and registering points distally on the femur, the hip offset, leg length can be identified and compared to pre-op or to what was planned on the GUI. Adjustments can be made to achieve optimal positioning and stability by adjusting the modular components (femoral head, neck, acetabular liner, depth of the cup or femoral stem). (See FIG. 5D (d)) The hip joint motion as well as stability can now be tested and tracked with the components reduced and the soft tissue tensioned appropriately.

In summary, described here is a highly accurate, ultrasonic-based, disposable navigation system that allows the sensorization of standard hip instruments and implants to provide the surgeon real time knowledge of implant positioning and the optimal leg length, offset, and joint stability, to achieve a successful joint procedure. The system can be integrated into a imageless or image free platform and incorporates wirelessly with a GUI that houses the necessary software for intra-op surgical adjustments. The mobility of the sensors, allows attachment to the bony pelvis and femur, or incorporated into the trials themselves. They can be attached to instruments controlled by the surgeon or a robotic haptic controlled instrumented arm. The data of the surgery can now be wirelessly sent to a data registry. Other sensory placement arrangements include, but are not limited to:
Sensorized acetabular outer shell trial or provisional component
Sensorized acetabular liner or insert trial or provisional component
Sensorized acetabular outer shell prosthesis
Sensorized acetabular liner or insert prosthesis
Sensorized acetabular non-modular prosthesis
Sensorized femoral trial or provisional component(s)
Sensorized femoral component(s)

FIG. 5B illustrates detection of femur head in the pelvis by way of circular motion about the pivot point. FIG. 5C demonstrates a method for prosthetic evaluation by calculating a first parameter set (offset and distance) of the first object with respect to the pivot point. The next steps can entail inserting a prosthetic in the first object (femur neck/head), calculating a second parameter set (offset and length) of the third object with respect to the pivot point. Report a differential between the first and second parameter set indicating changes in offset and length.

FIG. 5C illustrates detection of pre-surgery femur length and offset and post-surgery femur length and offset. FIG. 5D is a method for acetabular cup placement in accordance with one embodiment which can including mounting a first sensor 502 to the pelvis, and mounting a second sensor 501 to an impactor tool 503. The method can entail inserting the impactor tool in the acetabulum of the pelvis and rotating in an approximately circular pattern, tracking movement of impactor tool relative to pelvis and determine therefrom a location of the acetabulum. The method can further compare the location of the acetabulum during placement with respect to the pivot point previously determined from a sensorized natural leg movement. Upon insertion of an acetabular cup 105, the method can recalculate and compare the location of the acetabulum determined from sensorized leg movement and the pivot point previously determined from sensorized natural leg movement.

FIG. 5D further illustrates a sensorized hip impactor 503 with a positional sensor 501 and a cup 105 to be placed in the pelvis. Pelvic movement is compensated for via the sensor 502. As shown in (a), the target pelvis head can be predetermined from previous rotation. The impactor tool is guided and released in place as shown in (b). The method for acetabulum placement will utilize a sensorized acetabular outer shell trial (105) to assess optimal position of the prosthesis in the acetabulum with respect to vertical inclination and version. The optimal position can be defined as the position that provides the highest stability and resistance to dislocation.

A sensorized acetabular prosthesis may be utilized in this method where the sensorized acetabular prosthesis includes modular components and the outer shell is separate from an insert or liner comprising the articulation surface for articulation against a femoral head, and also the non-modular prosthesis where the acetabular outer shell and insert or liner comprise a single complete implant. A sensorized femoral component may be utilized in this method as well.

FIG. 5(D) (d) illustrates using a wand or probe 110 to register anatomical features in conjunction with the femur head ID. The wand 110 is used to register points that establish a length and offset from the femur head center 510 that helps define a reference frame for angular measurement: The wand (110) can be used to capture anatomical landmarks on the first object (pelvis), such as the left and right ASIS (anterior superior iliac spine) points, pubis points, or points inside the acetabulum. These landmarks can be used to define a coordinate system on the patient or to register the first object to a generic model in the case of imageless surgery or to a preoperative CT scan if there is preoperative imaging. If the patient is registered to a generic model or preoperative image, the coordinate system of the model or image can be used as the reference for measurement. The angular position (inclination and version) of the sensorized impactor tool can be reported relative to the reference frame created in a prior step. Embodiments herein apply to a femoral component or femoral components (plural to encompass modular components (e.g. femoral stems with modular necks), and “Sensorized acetabular non-modular prosthesis” to accommodate pre-assembled shell/liner design, use of which is facilitated by methods explained in the various embodiments herein.

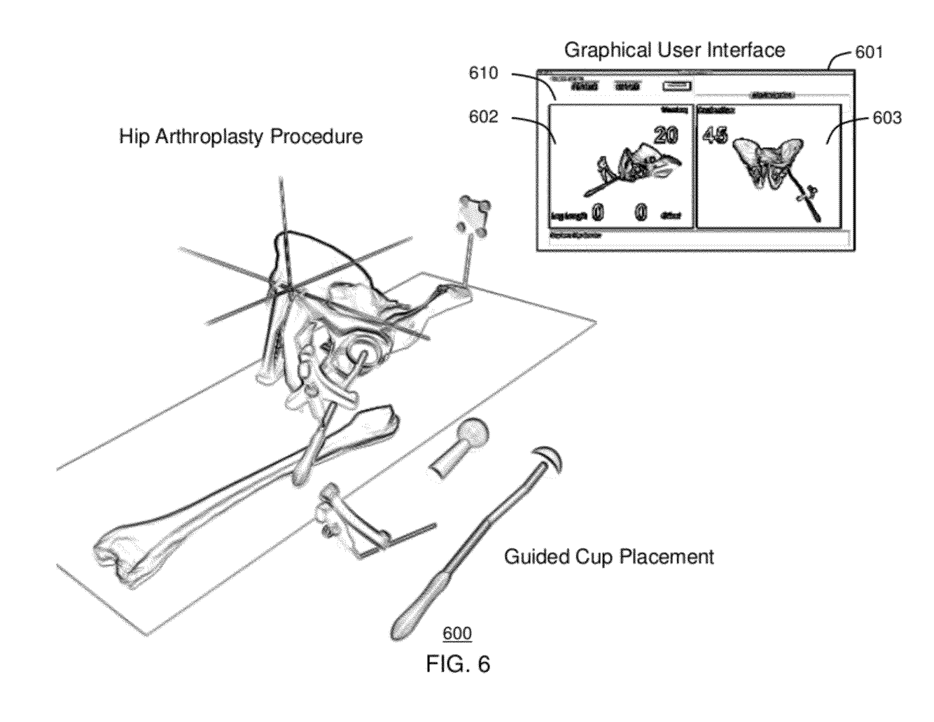

FIG. 6 depicts a system 600 having a presentation device 601 having a graphical user interface 610 having portions 602 and 603 that present version, inclination, depth, and position (and/or other parameters) as a particular sensoritized tool is being used or a bone element or prosthesis is being fitted or put into place during hip arthroplasty procedure for example.

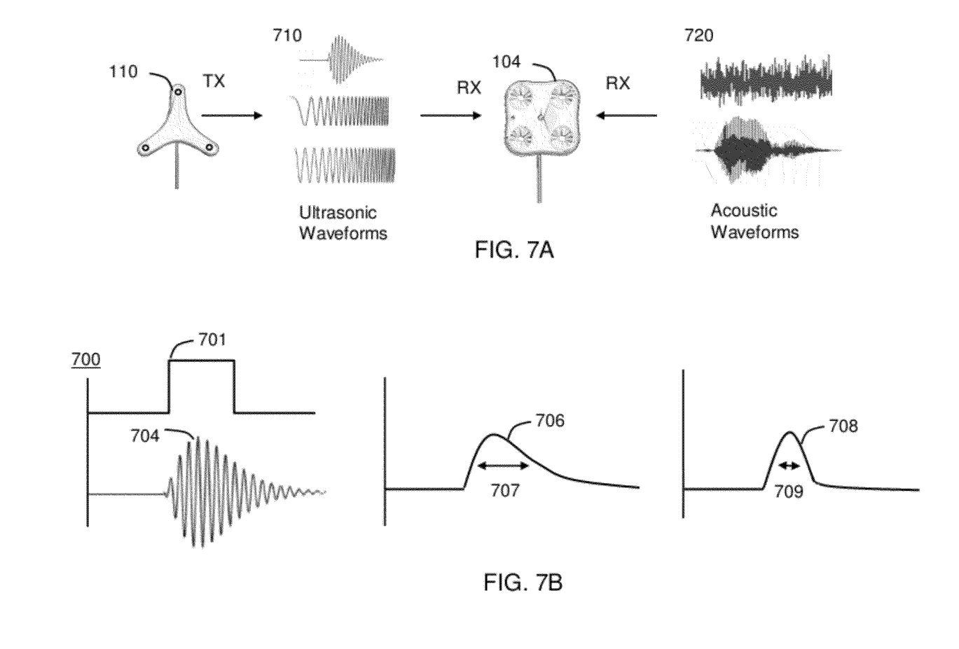

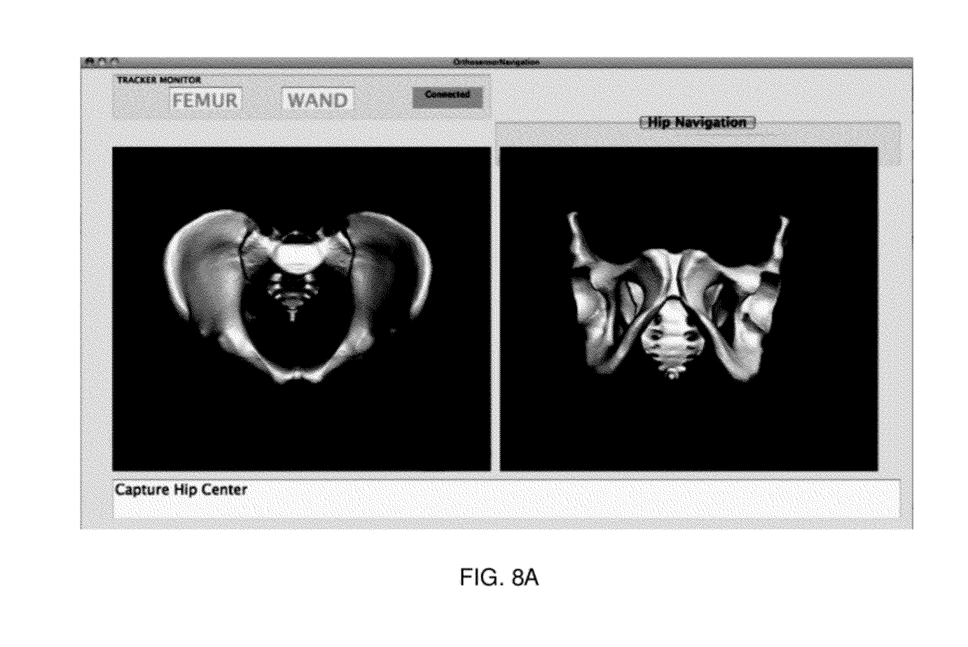

FIG. 7A depicts communication between exemplary components of the orthopedic tracking system in accordance with one embodiment. As illustrated, the transmitter 110 emits ultrasonic waveforms by way of three or more ultrasonic transducers on a probe in a three-dimensional sensing space. The receiver 104 by way of the four microphones captures the transmitted ultrasonic waveforms. As previously noted, a thermistor on the receiver measures ambient air temperature, which the processor uses to compensate for speed of sound.

Other sources of sound distortion may however be present during transmit and receiver operation of the tracking system, for example, voiced or noise signals in the operating environment. Thus, the microphones capture both ultrasonic and acoustic waveforms which are electrically converted to combined acoustic signals. In order to remove the external acoustic waveforms from the captured signal, the processor applies noise suppression and other digital filters to isolate the ultrasonic signals from the audio and noise signals.

During transmit-receive communications between a transmitter 110 and the receiver 220, the pod 102 digitally samples captured signals which as described above may be a combination of acoustic and ultrasonic waveforms to produce sampled received ultrasonic waveforms. The pod tracks a relative location and movement of the probe in the three-dimensional ultrasonic sensing space from differential time of flight waveform analysis of the sampled received ultrasonic waveforms. For precise tracking, the ultrasonic waveforms that overlap with digitally sampled acoustic waveforms received at the microphones are first isolated as indicated above through noise suppression and filtering, and thereafter, or in conjunction with, conditioned to suppress a ringing portion of the received ultrasonic waveforms. This signal conditioning minimizes a distortion associated with ultrasonic transducer ring-down during generation of a high-resolution position tracking of the probe.

FIG. 7B illustrates signal processing functions of this communication channel in accordance with one embodiment. As illustrated, a transmit pulse 601 sent to a transmitter 110 energizes one of the three ultrasonic transducers. The transducers in response generate an ultrasonic pulse 604 that is communicated through the air. The transducer is an electro-mechanical system that continues to ring even upon the end of the transmit pulse 701. Certain circuit configurations (RC, RLC) can selectively dampen the ringing responsive in a predetermined manner to received control information from the pod using microphone feedback in a closed loop configuration. The resonant fine structure of the pulse 704 is periodic based on the transmit frequency (e.g., 40 to 120 KHz). On receipt at the receiver 104, the processor applies an envelope function 706 with a main lobe width 707 that compresses the pulse shape 708 to a smaller width 709 without altering the resonant fine structure. Suppression of the ringing portion of the received ultrasonic waveforms that overlap with digitally sampled acoustic waveforms minimizes distortion associated with ultrasonic transducer ring-down during generation of a high-resolution position tracking of the probe. The pod 102 applies a weighting of a Time of Flight (TOF) ultrasonic distance measurement as a function of distance between the probe and the receiver. The weighting can be applied to an envelope of a received ultrasonic waveform for selective peak amplification. The pod 102 can also apply an acoustic spherical weighting within short range of the receiver approximately between 10 cm and 90 cm. The tracking performance improvement enhances user interface functionality, and accordingly, the systems ability to predict user interface commands or motion (e.g., circular patterns, line segments, range of motion) associated with operative workflow steps for presenting media.

FIG. 8 depicts an orthopedic alignment and balance GUI in accordance with one embodiment. As described above, upon the user pressing a switch 302 on the probe, the pod 102 directs a user command to the GUI 112 to alter a state of a user interface component as illustrated in FIG. 8. As an example, the GUI can illuminate an element of a hip such as a hip center, left ASIS, right ASIS, or Pubis as in FIG. 8(b) to indicate a next operation workflow step. The user, upon placement of a probe plate onto a desired measurement area, presses the center button of the switch 302 to capture the landmark plane, and the GUI marks component with a checkmark as in FIG. 8(c) to indicate successful capture. The GUI 112 can automatically scroll to the next GUI element such as the Left ASIS as in 8(d, e, and f) and indicate a successful capture. The GUI can further present various sensoritized tools or elements and represent them on the GUI 112 as the tools or elements are being manipulated as illustrated by the impactor tool shown in FIG. 8(g).

The illustrations of embodiments described herein are intended to provide a general understanding of the structure of various embodiments, and they are not intended to serve as a complete description of all the elements and features of apparatus and systems that might make use of the structures described herein. Many other embodiments will be apparent to those of skill in the art upon reviewing the above description. Other embodiments may be utilized and derived there from, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative rather than a restrictive sense.

Accordingly, this application also incorporates by reference the following Applications: U.S. Pat. No. 7,788,607 (Attorney Docket No. 80009) entitled “Method and System for Mapping Virtual Coordinates”, U.S. patent application Ser. No. 11/683,410 (Attorney Docket No. B0011) entitled “Method and System for Three-Dimensional Sensing”, U.S. patent application Ser. No. 11/683,412 (Attorney Docket No. B00.12 entitled “Application Programming Interface (API) for Sensory Events”, U.S. patent application Ser. No. 11/684,413 (Attorney Docket No. 800.13) entitled “Visual Toolkit for a Virtual User Interface”, U.S. patent application Ser. No. 11/683,415 (Attorney Docket No. B00.14) entitled “Virtual User Interface Method and Device Thereof”, U.S. patent application Ser. No. 11/683,416 (Attorney Docket No. B00.15) entitled “Touchless Tablet Method and Device Thereof” and U.S. patent application Ser. No. 12/050,790 (Attorney Docket No. 80023) entitled Method and Device for Touchless Media Searching.

Such embodiments of the inventive subject matter may be referred to herein, individually and/or collectively, by the term “invention” merely for convenience and without intending to voluntarily limit the scope of this application to any single invention or inventive concept if more than one is in fact disclosed. Thus, although specific embodiments have been illustrated and described herein, it should be appreciated that any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

These are but a few examples of embodiments and modifications that can be applied to the present disclosure without departing from the scope of the claims stated below. Accordingly, the reader is directed to the claims section for a fuller understanding of the breadth and scope of the present disclosure.

What is claimed is:

1. An apparatus, comprising:
a receiver that receives ultrasonic waveforms from a probe that emits the ultrasonic waveforms from three or more ultrasonic transducers in a three-dimensional sensing space; and
a controller coupled to a memory, wherein the memory comprises computer instructions which, when executed by the controller, cause the controller to:
digitally sample ultrasonic waveforms from the three or more microphones on the receiver to produce sampled received ultrasonic waveforms; and
track a relative location and movement of the probe in the three-dimensional ultrasonic sensing space from differential time of flight waveform analysis of the sampled received ultrasonic waveforms.

2. The apparatus of claim 1, wherein the computer instructions cause the controller to suppress a ringing portion of the received ultrasonic waveforms that overlap with digitally sampled acoustic waveforms received at the microphones.

3. The apparatus of claim 2, wherein the computer instructions cause the controller to minimize distortion associated with ultrasonic transducer ring-down during generation of a high-resolution position tracking of the probe.

4. The apparatus of claim 3, wherein the computer instructions cause the controller to further recognize an operation workflow and report measurement data from the probe associated with the operation workflow.

5. The apparatus of claim 1, wherein the computer instructions cause the controller to receive a user interface command from the probe during tracking associated with the high-resolution position tracking of the probe and present a media that corresponds to the user interface command, wherein the media is at least one among audio, image, video, and text.

6. The apparatus of claim 1, wherein the computer instructions cause the controller to present the media according to a customized use of the probe during an operation workflow.

7. The apparatus of claim 1, wherein the computer instructions cause the controller to navigate a menu system of a Graphical User Interface via the tracking of the probe relative to the receiver.

8. The apparatus of claim 1, wherein the computer instructions cause the controller to navigate a menu system of a Graphical User Interface by way of the probe.

9. The apparatus of claim 1, wherein the computer instructions cause the controller to weigh an envelope of a received ultrasonic waveform for selective peak amplification.

10. The apparatus of claim 1, wherein the probe is tracked in the three-dimensional sensing space relative to a stationary point on a bone having the receiver and wherein the computer instructions cause the controller to determine a location and relative displacement of the probe.

11. The apparatus of claim 10, wherein the bone is a hip bone and wherein the probe is used to capture a plurality of points relative to the stationary point to form an anatomical coordinate system.

12. The apparatus of claim 11, wherein the ultrasonic wand is used to capture at least a left anterior superior iliac spine (ASIS) point, a right ASIS point, and a pubis point on the hip bone.

13. The apparatus of claim 11, wherein the probe is affixed to a reaming device for guided reaming of an acetabulum of the hip bone.

14. The apparatus of claim 11, wherein the ultrasonic wand is affixed to a sensorized impactor for guided placement of a prosthesis-receiving cup within an acetabulum of the hip bone.

15. A probe, comprising:
three or more ultrasonic transducers that emit ultrasonic waveforms towards a receiver that receives the ultrasonic waveforms in a three-dimensional ultrasonic sensing space, wherein the receiver has a controller that digitally samples the ultrasonic waveforms from three or more microphones on the receiver to produce sampled received ultrasonic waveforms and tracks a relative location and movement of the probe in the three-dimensional ultrasonic sensing space from differential time of flight waveform analysis of the sampled received ultrasonic waveforms.

16. The probe of claim 15, wherein the probe is used for measuring an offset of a femur and any prosthetic insert from a hip bone and for measuring a length of the femur using relative distances from the receiver affixed in relation to the hop bone.

17. The probe of claim 15, wherein the bone is a hip bone and wherein the probe is an ultrasonic wand used in a posterior approach to capture at least one of a left ASIS point or a right ASIS point and capture a hip center by measuring an acetabulum of the hip bone.

18. A portable measurement system, comprising:
a probe comprising
a plurality of ultrasonic transducers that emit ultrasonic waveforms for creating a three-dimensional sensing space;
a user interface control that captures a location and position of the probe in the three-dimensional sensing space; and
a receiver comprising:
a plurality of microphones to capture the ultrasonic waveforms transmitted from the probe to produce captured ultrasonic waveforms; and
a digital signal processor that digitally samples the captured ultrasonic waveforms and tracks a relative location and movement of the probe with respect to the receiver in the three-dimensional ultrasonic sensing space from time of flight waveform analysis.

19. The portable measurement system of claim 18, wherein the receiver further comprises an inertial measurement that detects abrupt physical motion of a hand-operated tool coupled to the probe when exceeding threshold limits of a pre-specified plan

20. The portable measurement system of claim 19, wherein the inertial measurement unit comprises at least one of an accelerometer for measuring gravitational vectors, a magnetometer for measuring intensity of earth's magnetic field and corresponding north vector, a gyroscope for stabilizing absolute spatial orientation and position.
